SEQUENCE LISTING (75 sequences)

SEQ ID NO: 1 - protein, 112 aa, Synthetic construct
MPTTTTKVDIAAFDPDKDGTIDLKEALAAGSAAFDKLDPDKDGTLDAKELKGRVSEADLKKLDPDNDGTLDKKEYLAAVEAQFKAANPDNDGTIDARELASPAGSALVNLIR

SEQ ID NO: 2 - protein, 532 aa, Synthetic construct
MPTTTTKVDIAAFDPDKDGTIDLKEALAAGSAAFDKLDPDKDGTLDAKELKGRVSEADLKKLDPDNDGTLDKKEYLAAVEAQFKAANPDNDGTIDARELASPAGSALVNLIRAAASGGGENLYFQGAETVESCLAKPHTENSFTNVWKDDKTLDRYANYEGCLWNATGVVVCTGDETQCYGTWVPIGLAIPENEGGGSEGGGSEGGGSEGVGTKPPEYGDTPIPGYTYINPLDGTYPPGTEQNPANPNPSLEESQPLNTFMFQNNRFRNRQGALTVYTGTVTQGTDPVKTYYQYTPVSSKAMYDAYWNGKFRDCAFHSGFNEDPFVCEYQGQSSDLPQPPVNAGGGSGGGSGGGSEGGGSEGGGSEGGGSEGGGSGGGSGSGDFDYEKMANANKGAMTENADENALQSDAKGKLDSVATDYGAAIDGFIGDVSGLANGNGATGDFAGSNSQMAQVGDGDNSPLMNNFRQYLPSLPQSVECRPYVFGAGKPYEFSIDCDKINLFRGVFAFLLYVATFMYVFSTFANILRNKES

SEQ ID NO: 3 - protein, 546 aa, synthetic construct
MPTTTTKVDIAAFDPDKDGTIDLKEALAAGSAAFDKLDPDKDGTLDAKELKGRVSEADLKKLDPDNDGTLDKWEYLAAVEAQFKAANPDNDGTIDARELASPAGSALVNLIRAAASGGGENLYFQGGGGSGAPENLYFQGAETVESCLAKPHTENSFTNVWKDDKTLDRYANYEGCLWNATGVVVCTGDETQCYGTWVPIGLAIPENEGGGSEGGGSEGGGSEGVGTKPPEYGDTPIPGYTYINPLDGTYPPGTEQNPANPNPSLEESQPLNTFMFQNNRFRNRQGALTVYTGTVTQGTDPVKTYYQYTPVSSKAMYDAYWNGKFRDCAFHSGFNEDPFVCEYQGQSSDLPQPPVNAGGGSGGGSGGGSEGGGSEGGGSEGGGSEGGGSGGGSGSGDFDYEKMANANKGAMTENADENALQSDAKGKLDSVATDYGAAIDGFIGDVSGLANGNGATGDFAGSNSQMAQVGDGDNSPLMNNFRQYLPSLPQSVECRPYVFGAGKPYEFSIDCDKINLFRGVFAFLLYVATFMYVFSTFANILRNKES

SEQ ID NO: 4 - protein, 121 aa, synthetic construct
MPTTTTKVDIAAFDPDKDGTIDLKEALAAGSAAFDKLDPDKDGTLDAKELKGRVSEADLKKLDPDNDKKEYLAAVEAQFKAANPDNDGTIDARELASPAGSALVNLIRAAASGGGENLYFQ

SEQ ID NO: 5 - protein, 73 aa, synthetic construct
MKKSLVLKASVAVATLVPMLSFAAEGDDPAKAAFNSLQASATEYIGYAWAMVVVIVGATIGIKLFKKFTSKAS

SEQ ID NO: 6 - protein, 85 aa, synthetic construct
DPDNDGTLDKKEMKKSLVLKASVAVATLVPMLSFAAEGDDPAKAAFNSLQASATEYIGYAWAMVVVIVGATIGIKLFKKFTSKAS

SEQ ID NO: 7 - protein, 85 aa, synthetic construct
DPDNDGTLDKWEMKKSLVLKASVAVATLVPMLSFAAEGDDPAKAAFNSLQASATEYIGYAWAMVVVIVGATIGIKLFKKFTSKAS

SEQ ID NO: 8 - protein, 6 aa, synthetic construct
ENLYFQ

SEQ ID NO: 9 - protein, 12 aa, synthetic construct
DPDKDGTIDLKE

SEQ ID NO: 10 - protein, 12 aa, synthetic construct
DPDKDGTLDAKE

SEQ ID NO: 11 - protein, 12 aa, synthetic construct
DPDNDGTLDKKE

SEQ ID NO: 12 - protein, 12 aa, synthetic construct
DPDNDGTLDKWE

SEQ ID NO: 13 - protein, 530 aa, Synthetic construct
MGSSHHHHHHSSGLVPRGSHMVSKGEELFTGVVPILVELDGDVNGHKFSVSGEGEGDATYGKLTLKFICTTGKLPVPWPTLVTTLTWGVQCFARYPDHMKQHDFFKSAMPEGYVQERTIFFKDDGNYKTRAEVKFEGDTLVNRIELKGIDFKEDGNILGHKLEYNAISDNVYITADKQKNGIKANFKIRHNIEDGSVQLADHYQQNTPIGDGPVLLPDNHYLSTQSALSKDPNEKRDHMVLLEFVTAAGITLSMDELYKGDPDNDGTLDKKEMVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSALSKDPNEKRDHMVLLEFVTAAGITLGMDELYKLEDPAANKAITLGMDELYK

SEQ ID NO: 14 - protein, 12 aa, Synthetic construct
TWFLGSCLSMTG

SEQ ID NO: 15 - protein, 12 aa, Synthetic construct
GGVVPVSKYMDE

SEQ ID NO: 16 - protein, 12 aa, Synthetic construct
VGVCCAWFRNFG

SEQ ID NO: 17 - protein, 12 aa, Synthetic construct
HDTTVCSGSLAL

SEQ ID NO: 18 - protein, 12 aa, Synthetic construct
LTWDIDDCPAPG

SEQ ID NO: 19 - protein, 12 aa, Synthetic construct
LSKFLMGFFWIL

SEQ ID NO: 20 - protein, 12 aa, Synthetic construct
LILVSELHSSRL

SEQ ID NO: 21 - protein, 12 aa, Synthetic construct
SSSGSNLVVFEY

SEQ ID NO: 22 - protein, 12 aa, Synthetic construct
FFAASLFSKNRY

SEQ ID NO: 23 - protein, 12 aa, Synthetic construct
FIMLAHWLLWIL

SEQ ID NO: 24 - protein, 12 aa, Synthetic construct
FLFSMYRVFGPQ

SEQ ID NO: 25 - protein, 12 aa, Synthetic construct
LFRYFLGFTFYF

SEQ ID NO: 26 - protein, 12 aa, Synthetic construct
WKVSSFFVDGFC

SEQ ID NO: 27 - protein, 12 aa, Synthetic construct
MSKWFDSFRSEG

SEQ ID NO: 28 - protein, 12 aa, Synthetic construct
MLSFLREFPDAF

SEQ ID NO: 29 - protein, 12 aa, Synthetic construct
YSSLKFLVVSGA

SEQ ID NO: 30 - protein, 12 aa, Synthetic construct
LMAWYCWYMGVR

SEQ ID NO: 31 - protein, 12 aa, Synthetic construct
FVVGQSQNTQFA

SEQ ID NO: 32 - protein, 12 aa, Synthetic construct
HIMGVFSLSVGS

SEQ ID NO: 33 - protein, 12 aa, Synthetic construct
STFRLIARVLRW

SEQ ID NO: 34 - protein, 12 aa, Synthetic construct
HSILGISGVSNV

SEQ ID NO: 35 - protein, 12 aa, Synthetic construct
ENLVNYCWCMSI

SEQ ID NO: 36 - protein, 12 aa, Synthetic construct
RKLGGMIGYWTS

SEQ ID NO: 37 - protein, 12 aa, Synthetic construct
ASNELKGSSMFG

SEQ ID NO: 38 - protein, 12 aa, Synthetic construct
FCIPGCLNFFIV

SEQ ID NO: 39 - protein, 12 aa, Synthetic construct
LCLFLRLVDVSL

SEQ ID NO: 40 - protein, 12 aa, Synthetic construct
YSSLKFLVVSGA

SEQ ID NO: 41 - protein, 12 aa, Synthetic construct
KMVPWGSVIWFG

SEQ ID NO: 42 - protein, 12 aa, Synthetic construct
VHVFMRRFGVLW

SEQ ID NO: 43 - protein, 12 aa, Synthetic construct
YTSGVRYVLWWW

SEQ ID NO: 44 - protein, 12 aa, Synthetic construct
CYLCRAFPNFGS

SEQ ID NO: 45 - protein, 12 aa, Synthetic construct
GSRLLACDFFLY

SEQ ID NO: 46 - protein, 12 aa, Synthetic construct
ILLMSCRGCVKS

SEQ ID NO: 47 - protein, 12 aa, Synthetic construct
TAPHCSLGARNI

SEQ ID NO: 48 - protein, 12 aa, Synthetic construct
TLEFLLSPCQLA

SEQ ID NO: 49 - protein, 12 aa, Synthetic construct
YRSSLHLFSSLS

SEQ ID NO: 50 - protein, 12 aa, Synthetic construct
WTFDSLDSSTCG

SEQ ID NO: 51 - protein, 12 aa, Synthetic construct
LQFILCAKGDDV

SEQ ID NO: 52 - protein, 12 aa, Synthetic construct
QCMRLSWVNNCV

SEQ ID NO: 53 - protein, 12 aa, Synthetic construct
HFFLLANTTVVR

SEQ ID NO: 54 - protein, 12 aa, Synthetic construct
FYGLWFYRMSGS

SEQ ID NO: 55 - protein, 12 aa, Synthetic construct
LDIIKRFCLGMH

SEQ ID NO: 56 - protein, 12 aa, Synthetic construct
MGSVRSDRYQFV

SEQ ID NO: 57 - protein, 12 aa, Synthetic construct
NITLVKSILVDR

SEQ ID NO: 58 - protein, 12 aa, Synthetic construct
FHTSSEFLSSVC

SEQ ID NO: 59 - protein, 12 aa, Synthetic construct
TGNIHGSGLLLR

SEQ ID NO: 60 - protein, 13 aa, Synthetic construct
DTNNDGEYEGDEL

SEQ ID NO: 61 - protein, 13 aa, Synthetic construct
DTNNDGLYEGDEL

SEQ ID NO: 62 - protein, 13 aa, Synthetic construct
DTNNDGGYEGDEL

SEQ ID NO: 63 - protein, 13 aa, Synthetic construct
DTNNDGRYEGDEL

SEQ ID NO: 64 - protein, 13 aa, Synthetic construct
DTNNDGSYEGDEL

SEQ ID NO: 65 - protein, 13 aa, Synthetic construct
DTNNDGSYEGDEL

SEQ ID NO: 66 - protein, 13 aa, Synthetic construct
DTNNDGTYEGDEL

SEQ ID NO: 67 - protein, 13 aa, Synthetic construct
DTNNDGVYEGDEL

SEQ ID NO: 68 - protein, 13 aa, Synthetic construct
DTNNDGIYEGDEL

SEQ ID NO: 69 - protein, 17 aa, Synthetic construct
YIDTNNDGWYEGDELLA

SEQ ID NO: 70 - protein, 17 aa, Synthetic construct
ACVDWNNDGWYEGDECA

SEQ ID NO: 71 - protein, 14 aa, Synthetic construct
DKDGDGTITTKELG

SEQ ID NO: 72 - protein, 14 aa, Synthetic construct
DKDGDDTITTKELG

SEQ ID NO: 73 - protein, 12 aa, synthetic construct
NPDNDGTIDARE

SEQ ID NO: 74 - protein, 17 aa, synthetic construct
YIDTNNDGWYEGDELLA

SEQ ID NO: 75 - protein, 17 aa, synthetic construct
ACVDWNNDGWYEGDECA

(19) United States
(12) Patent Application Publication
(10) Pub. No.: US 2024/0167118 A1
(43) Pub. Date: May 23, 2024

(54) Methods and Preparation of M13 Phage for Passive Recovery of Rare Earth Elements

(71) Applicant: The Government of the United States of America, as represented by the Secretary of the Navy, Arlington, VA (US)

(72) Inventors: Jinny L. Liu, Ellicott City, MD (US); Patricia M. Legler, Derwood, MD (US); Keith Whitener, Alexandria, VA (US); Steven Bennett, Owings, MD (US)

(21) Appl. No.: 18/516,430

(22) Filed: Nov. 21, 2023

Related U.S. Application Data
(60) Provisional application No. 63/384,703, filed on Nov. 22, 2022.

(51) Int. Cl. C22B 3/18; C12N 7/00; C22B 59/00

(52) U.S. Cl. CPC C22B 3/18; C12N 7/00; C22B 59/00; C12N 2795/14121; C12N 2795/14152

(57) ABSTRACT

A construct comprising an M13 phage displaying one or more metal-binding protein sequences on capsid proteins (for example metal binding protein sequences from Lanmodulin) or on the tail protein (pIII) is effective to bind and release rare earth metal (REE) for the extraction, concentration, and/or purification thereof.

### CROSS-REFERENCE TO RELATED APPLICATIONS

This application claims the benefit of U.S. Provisional Patent Application No. 63/384,703 filed on Nov. 22, 2022, which is incorporated herein by reference in its entirety.

### FEDERALLY-SPONSORED RESEARCH AND DEVELOPMENT

The United States Government has ownership rights in this invention. Licensing inquiries may be directed to Office of Technology Transfer, US Naval Research Laboratory, Code 1004, Washington, DC 20375, USA; +1.202.767.7230; techtran@nrl.navy.mil, referencing NC 211340.

### INCORPORATION BY REFERENCE

This Application incorporates by reference the Sequence Listing XML file submitted herewith via the patent office electronic filing system having the file name “211340US2-sequences.xml” and created on Nov. 21, 2022 with a file size of 68,201 bytes.

### BACKGROUND

In order to extract rare earth elements, current hydrometallurgical methods use extractants such as CYANEX 923 in organic solvents. The metals are acid-extracted from the organic phase after other impurities are removed. Such solvent extraction methods generate large volumes of liquid waste and suffer from lack of specificity while posing challenges for purification and separation when performed on the industrial scale.

A need exists for new methods for recovery of rare earth elements.

### BRIEF SUMMARY

In a first embodiment, a biological construct for capturing rare earth elements comprises an M13 phage displaying one or more metal-binding protein sequences on capsid proteins thereof.

In another embodiment, a method of capturing a rare earth element includes providing a construct according to the first embodiment; contacting, under acidic conditions, the construct with a solution comprising a rare earth metal (REE), wherein the construct and the solution are initially separated by a dialysis membrane; allowing the REE to pass through the membrane and bind to the construct at low pH of about 4 to 6; and eluting, under neutral or acid pH conditions, optically in a glycine buffer of about pH 1.4), the REE from the construct.

A further embodiment is a biological construct comprising one or more amino acid sequences selected from the group consisting of SEQ ID NOs: 9 to 70.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A and 1B depict M13 filamentous phage structure. FIG. 1A depicts LanM and its derivative displayed through tail protein (PIII). FIG. 1B shows EF peptide (12-14 mers) displayed on the major capsid protein (PVIII).

FIGS. 2A and 2B provide an assessment of isolated display phages. FIG. 2A shows the gel stained with DNA staining dye Cyber Safe while FIG. 2B depicts the same gel stained with GelCode™ blue protein dye (Thermofisher). The phages and DNA were loaded into 1% Tris Acetate EDTA agarose gel and separated by passing a current at 100 V for 1 hrs. The labels for each lane are as follows: M for 1 kb plus DNA marker, 1 for 0.4 μg of LanMpEcan21 DNA, 2 for 50 μg of LanM4 ϕ, 3 for 50 μg of LanM5 WT ϕ, 4 for 0.4 μg of p8-p21 DNA, 5, for 50 μg of p8 WT ϕ, 6 for 50 μg of K94Wp8 ϕ, and 7 for 30 μg of M13K07 ϕ.

### DETAILED DESCRIPTION

### Definitions

Before describing the present invention in detail, it is to be understood that the terminology used in the specification is for the purpose of describing particular embodiments, and is not necessarily intended to be limiting. Although many methods, structures and materials similar, modified, or equivalent to those described herein can be used in the practice of the present invention without undue experimentation, the preferred methods, structures and materials are described herein. In describing and claiming the present invention, the following terminology will be used in accordance with the definitions set out below.

As used herein, the singular forms “a”, “an,” and “the” do not preclude plural referents, unless the content clearly dictates otherwise.

As used herein, the term “and/or” includes any and all combinations of one or more of the associated listed items.

As used herein, the term “about” when used in conjunction with a stated numerical value or range denotes somewhat more or somewhat less than the stated value or range, to within a range of ±10% of that stated.

### Overview

Peptides and proteins can be used for the binding and purification of rare earth elements (REE). This includes the elements of yttrium, scandium, and/or the lanthanoid (lanthanide) element of lantahum (La) through Lutetium (Lu) on the periodic table. In some cases these proteins/peptides could also bind actinoids (actinides).

Peptides can be produced by bacteria or they can be chemically synthesized; however, chemical synthesis requires solvents and costly purification. Small peptides are difficult to re-capture once they are dissolved in a large volume of liquid. Their small size means that they must be anchored to a resin, bead or a biomaterial (e.g. phage, bacterium) so that they are not lost in continuous flow devices or large volumes of aqueous solutions. Several immobilization strategies for biomolecules have been proposed such as immobilizing phage to paper (see ref. 1) or anchoring them to resins (see ref. 2). Those involved in hydrometallurgy are typically unfamiliar with methods to create or handle these biomaterials so success in such applications has remained elusive. Moreover, feed stock material may consist of coal ash, mud, electronic waste (eWaste), acid mine drainage, or pulverized ores soaked in acid. The presence of bacteria and proteases in the feedstock material can damage some of these biomaterials. The tolerances of biomaterials to pH and temperature extremes also impose challenges.

Engineered phage and bacterial spores have been proposed as possible biomaterials for REE purification since these can be generated with renewable resources with no significant detriment to the environment (e.g. glycerol, yeast extract, tryptone, phosphate, lactose, glucose, etc.) and can provide specificity. Biomaterials such as regenerated cellulose dialysis membranes can also provide resistance to protease digestion. Biomaterials such as fungi, bacteria, or viruses can be selected or made resilient to extreme pH and temperature. While the cost of disposal of organic chemicals and waste can affect the profitability of mining these high-value metal ions, biomaterials can be disposed of after heat sterilization with minimal impact on the environment, making them amenable to industrial operations.

A protein similar to calmodulin, called Lanmodulin, was reported in 2019 by Cotruvo, et al. (see ref. 8). It was shown to bind calcium and other REE with picomolar dissociation constants (see ref. 3). Peptides and lanthanum binding tags (LBT) have also been shown to bind REE (see ref. 4) such as Nd and Tb in the uM to nM range.

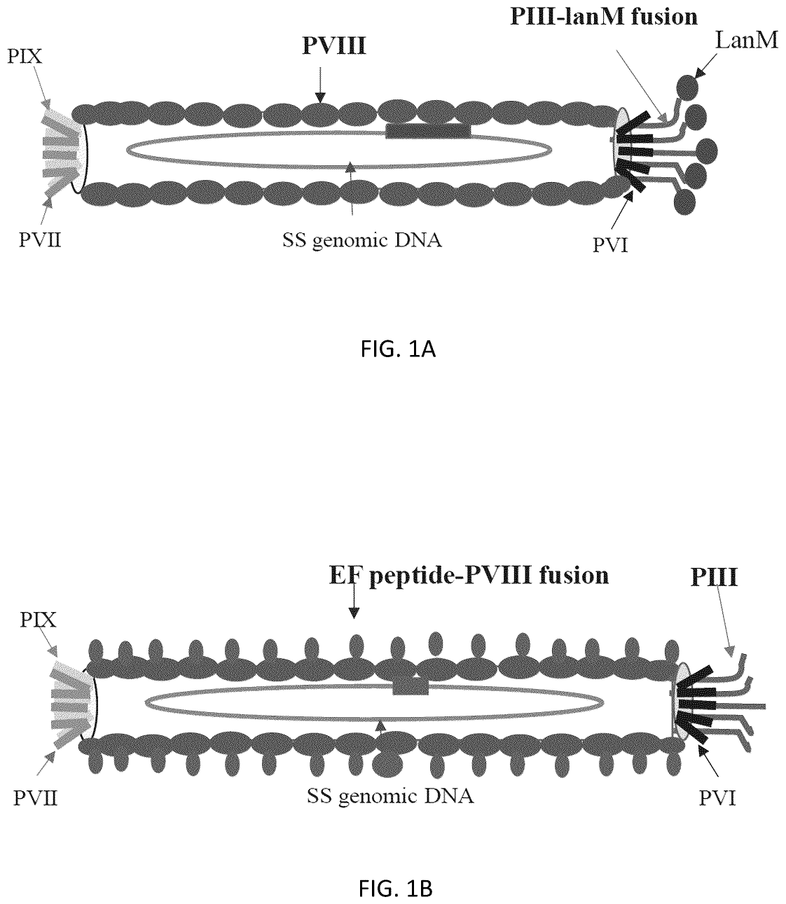

Peptides can be displayed at two locations on filamentous phage: the tail protein called PIII or on the capsid protein called PVIII (73 amino acids), as seen in FIGS. 1A and 1B. Only 5 copies of the tail protein are present per phage whereas approximately 2700 capsid proteins are present per phage. The high density of the capsid proteins is attractive since considerably less biomaterial would be needed to purify a given quantity of metal; however only very short sequences are tolerated by the phage and sequences which are too long can compromise the assembly of the phage particle. Thus, successfully genetically engineering a sequence into a phage capsid or selecting a REE-binding phage from a phage display library calls for empirical testing.

One can estimate how much metal each biomaterial can hold based upon the MW and number of metal binding sites. One can also estimate the volume of bacterial media required to produce the biomaterials sufficient to bind 1 g of the REE (Nd). Table 1 shows these estimates for four biomaterials: purified LanM protein, tail-protein displayed LanM, capsid-displayed peptides, and the Bacillus spores described in Dong et al. (ref. 5).

TABLE 1
Calculated or reported capacities of biomaterials for REE purification.

| | Molar capacity | Mass capacity | Quantity of | |
|---|---|---|---|---|
| | of 1 mg of for | of 1 mg of | material to | Volume of media |
| | material for | material for | capture 1 g of | needed to make the |
| Biomaterial | Nd³⁺ (nmol) | Nd³⁺ (mg) | Nd³⁺ (grams) | biomaterial (liters) |
| LanM pIII tail phage | 0.55 | 0.000080 | 12,500 | 62,500 |
| (SEQ ID NOs: 2, 3) | | | | |
| LanM purified protein | 240 | 0.034 | 29 | 2,041 |
| (SEQ ID NO: 1) | | | | |
| LanM pVIII capsid phage | 159 | 0.023 | 43.5 | 20 to 218 |
| (SEQ ID NOs: 5, 6) | | | | |
| Bacillus spores of ref. 5 | 100-200 | 0.015-0.029 | 34.7-69.3 | unknown |

As described herein, genetically engineered M13 phages were constructed displaying Lanmodulin (LanM) on the tail protein PIII (SEQ ID NOs: 2, 3] and M13 phages displaying peptides on the PVIII capsid protein using an phagemid or recombinant phage (SEQ ID NOs: 6, 7). Also developed was a method for utilizing this material for REE purification.

Dialysis bags were used here to retain the REE-binding phage. Metal ions can freely pass through the regenerated cellulose membrane. The cellulose dialysis bags are also a biomaterial. The advantage of using a dialysis bag is that no immobilization step was required, and it could be physically removed from aqueous solutions. Immobilization steps can require additional chemistry. It also restricted access to the REE-binding biomaterial by bacteria, proteases, or debris. The molecular weight (MW) cutoff (MWCO) of the dialysis membrane was selected to allow passage of ions but not the phage biomaterial. For example, although peptides alone can bind REE, they likely would have been lost during the dialysis: the MW of the Lanmodulin EF3 peptide described below is 1,346 Da and so it would not be retained. The 3,500 MWCO used in the examples was effective in retaining the construct having peptides displayed on phage capsid proteins.

### EXAMPLES

Cloning of phagemid for tall and capsid display vectors. The coding sequence was cloned into a pecan21 phagemid display vector (a gift from Dr. Andrew Hayhurst, Texas Biomedical Research Institute). The phagemid contains the gene III sequence. The N-terminus for the fusion protein construct contains a cloning site and ENLYFQ sequence (SEQ ID NO: 8) corresponding to a TEV (tobacco etch virus) protease cleavage site. The capsid display phagemid was newly developed by replacing gene III sequence with major capsid gene VIII. LanM has four metal binding peptides DPDKDGTIDLKE (EF1) (SEQ ID NO: 9), DPDKDGTLDAKE (EF2) (SEQ ID NO: 10), DPDNDGTLDKKE (EF3) (SEQ ID NO: 11), NPDNDGTIDARE (SEQ ID NO: 73) which are thought to coordinate the metal ions (PDB 6MI5 and 8FNS) (ref. 6). LanM has been shown to have picomolar affinity for REE (3). Here, LanM was displayed through the tail protein PIII (FIG. 1A), while the EF3 peptide and a tryptophan mutant thereof (termed EF3 K94W), DPDNDGTLDKWE (SEQ ID NO: 12) were displayed through the capsid protein PVIII (FIG. 1B). Other peptide sequences were also displayed for method development such as YIDTNNDGWYEGDELLA (SEQ ID NO: 74) and ACVDWNNDGWYEGDECA (SEQ ID NO: 75) (see ref. 9).

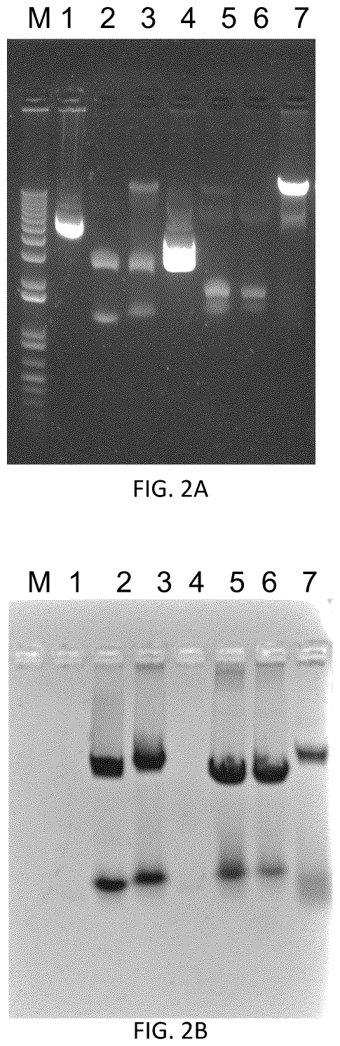

Preparation of display phages. Phagemid DNA with LanM/EF peptides inserted were transformed into XL1 blue on LB agar plates supplemented with 100 μg/mL Ampicillin (Amp) and 15 μg/mL Tetracycline (Tet). A single colony was grown overnight at 37° C. overnight in LB supplemented with Amp and Tet. Next day, 350 μL overnight culture was diluted into 10 mL LB culture with antibiotics and grew at 37° C. for approximately 2 hrs to reach log phase (OD600=0.50). The amount of M13K07 helper phages (New England Biolabs) with multiplicity of infection 20 to total bacteria were then added into bacterial culture and incubate at 37° C. for 30 min before spinning down cell pellet at 6000×g for 10 min at 15° ° C. The cell pellet was resuspended in 50 mL of LB supplemented with 100 μg/mL Amp and 25 μg/mL Kanamycin and the bacterial culture was then shaking in a speed of 250 rpm at 30° C. overnight. The following day the supernatant was collected after pelleting down the cells at 15,000×g for 10 min. Phages in supernatant were then precipitated down by adding 10 mL of 20% PEG8000 and 2.5 M NaCl into 50 mL supernatant and incubating on ice for at least an hour with occasional vigorous shaking. The phage supernatant was then pelleted down at 20,000×g for 30 min and resuspended in 2 mL 50 mM HEPES buffer (pH7.0). The phage suspension was subjected to another spin at 15,000 rpm for 5 min to further remove the undissolved particles before filtering through 0.22 μm membrane. The phage concentration was estimated by reading the absorbance at 280 nm for protein and the factor of 3.69E+13 plaque forming units (PFU) per mg protein. The isolated phages were assessed in a 1% Tris-acetate-EDTA (TAE) agarose gel with results shown in FIGS. 2A and 2B.

Cloning of recombinant M13 phage genome and preparation of recombinant M13 phages. The M13 helper genome operated as a template to replace pIII with LanM-pIII fusion and also to insert f1 ori to obtain LanM M13 recombinant phage. Using the same strategy, recombinant EF-p8 M13 phage, EF and its derivative-p8 DNA fragments are expected to be obtained by replacing p8 DNA in the genome and p15A ori with f1 ori DNA. Recombinant M13 phage genome can be prepared using non-host E. coli bacteria, such as 10beta, followed by transforming the genomic DNA into host bacteria, such as TG1 and XL1 blue. Plaque PCR assay can be used to amplify the fusion DNA for sequencing confirmation. M13 phages can then be produced in these M13 phage hosts and purified. Advantages of recombinant M13 phages are easily scaled to large scale production and produced homogenously with displayed proteins/peptides.

TABLE 2
Compositions of Engineered M13 phage proteins.

| | |
|---|---|
| Lanmodulin | MPTTTTKVDIAAFDPDKDGTIDLKEALAAGSAAFDKLDPDKDGTLDA |
| | KELKGRVSEADLKKLDPDNDGTLDKKEYLAAVEAQFKAANPDNDG |
| | TIDARELASPAGSALVNLIR- (SEQ ID NO: 1) |
| LanM5 | MPTTTTKVDIAAFDPDKDGTIDLKEALAAGSAAFDKLDPDKDGTLDA |
| (WT) pIII | KELKGRVSEADLKKLDPDNDGTLDKKEYLAAVEAQFKAANPDNDG |
| fusion | TIDARELASPAGSALVNLIRAAASGGGENIYRGAETVESCLAKPHTE |
| | NSFTNVWKDDKTLDRYANYEGCLWNATGVVVCTGDETQCYGTWV |
| | PIGLAIPENEGGGSEGGGSEGGGSEGVGTKPPEYGDTPIPGYTYINPLD |
| | GTYPPGTEQNPANPNPSLEESQPLNTFMFQNNRFRNRQGALTVYTGT |
| | VTQGTDPVKTYYQYTPVSSKAMYDAYWNGKFRDCAFHSGFNEDPF |
| | VCEYQGQSSDLPQPPVNAGGGSGGGSGGGSEGGGSEGGGSEGGGSE |
| | GGGSGGGSGSGDFDYEKMANANKGAMTENADENALQSDAKGKLDS |
| | VATDYGAAIDGFIGDVSGLANGNGATGDFAGSNSQMAQVGDGDNSP |
| | LMNNFRQYLPSLPQSVECRPYVFGAGKPYEFSIDCDKINLFRGVFAFL |
| | LYVATFMYVFSTFANILRNKES- (SEQ ID NO: 2) |
| K94W | MPTTTTKVDIAAFDPDKDGTIDLKEALAAGSAAFDKLDPDKDGTLDA |
| LanM pIII | KELKGRVSEADLKKLDPDNDGTLDKWEYLAAVEAQFKAANPDNDG |
| fusion | TIDARELASPAGSALVNLIRAAASGGGENLYFQGGGGSGAPENI YFQ |
| | GAETVESCLAKPHTENSFTNVWKDDKTLDRYANYEGCLWNATGVV |
| | VCTGDETQCYGTWVPIGLAIPENEGGGSEGGGSEGGGSEGVGTKPPE |
| | YGDTPIPGYTYINPLDGTYPPGTEQNPANPNPSLEESQPLNTFMFQNN |
| | RFRNRQGALTVYTGTVTQGTDPVKTYYQYTPVSSKAMYDAYWNGK |
| | FRDCAFHSGFNEDPFVCEYQGQSSDLPQPPVNAGGGSGGGSGGGSEG |
| | GGSEGGGSEGGGSEGGGSGGGSGSGDFDYEKMANANKGAMTENAD |
| | ENALQSDAKGKLDSVATDYGAAIDGFIGDVSGLANGNGATGDFAGS |
| | NSQMAQVGDGDNSPLMNNFRQYLPSLPQSVECRPYVFGAGKPYEFSI |
| | DCDKINLFRGVFAFLLYVATFMYVESTFANILRNKES- (SEQ ID NO: 3) |
| LanM4 | MPTTTTKVDIAAFDPDKDGTIDLKEALAAGSAAFDKLDPDKDGTLDA |
| (mutant) | KELKGRVSEADLKKLDPDNDKKEYLAAVEAQFKAANPDNDGTIDAR |
| pIII fusion | ELASPAGSALVNLIRAAASGGGENLYFQ |
| | GAETVESCLAKPHTENSFTNVWKDDKTLDRYANYEGCLWNATGVV |
| | VCTGDETQCYGTWVPIGLAIPENEGGGSEGGGSEGGGSEGVGTKPPE |
| | YGDTPIPGYTYINPLDGTYPPGTEQNPANPNPSLEESQPLNTFMFQNN |
| | RFRNRQGALTVYTGTVTQGTDPVKTYYQYTPVSSKAMYDAYWNGK |
| | FRDCAFHSGFNEDPFVCEYQGQSSDLPQPPVNAGGGSGGGSGGGSEG |
| | GGSEGGGSEGGGSEGGGSGGGSGSGDFDYEKMANANKGAMTENAD |
| | ENALQSDAKGKLDSVATDYGAAIDGFIGDVSGLANGNGATGDFAGS |
| | NSQMAQVGDGDNSPLMNNFRQYLPSLPQSVECRPYVFGAGKPYEFSI |
| | DCDKINLFRGVFAFLLYVATFMYVFSTFANILRNKES- |
| | (SEQ ID NO: 4) |
| pVIII | MKKSLVLKASVAVATLVPMLSFAAEGDDPAKAAFNSLQASATEYIG |
| | YAWAMVVVIVGATIGIKLFKKFTSKAS- |
| | (SEQ ID NO: 5) |
| EF3-pVIII | DPDNDGTLDKKEMKKSLVLKASVAVATLVPMLSFAAEGDDPAKAA |
| | FNSLQASATEYIGYAWAMVVVIVGATIGIKLFKKFTSKAS- |
| | (SEQ ID NO: 6) |
| EF3-K94W- | DPDNDGTLDKWEMKKSLVLKASVAVATLVPMLSFAAEGDDPAKAA |
| pVIII | FNSLQASATEYIGYAWAMVVVIVGATIGIKLFKKFTSKAS- |
| | (SEQ ID NO: 7) |

REE Purification Using M13 Phage. First, phage was prepared by placing 5 mg of phage into dialysis bags (SpectraPor, #133116, flat width 10 mm, regenerated cellulose, 3500 MWCO). The phage were dialyzed against 10 mL of 50 mM HEPES pH 7.0, 150 mM NaCl with 0.1 g Chelex resin outside of the dialysis bag. Next, REE was bound: NdFeB powder (16.7 mg/mL) dissolved in 6.8% nitric acid was diluted to 2 mg/mL with 50 mM HEPES, 150 mM NaCl and had a final pH 3.7. Dialysis bags containing the phages were placed in 7 mL of the 2 mg/mL NdFeB solution and allowed to bind the Nd³⁺ at room temperature (23±3° C.). After overnight incubation Fe oxides precipitated. Finally, REE was eluted: bags were then moved into 10 mL of 50 mM HEPES pH 7.0, 150 mM NaCl for 1 h at room temperature. Precipitate formed at the bottom of the tube. Elemental analysis of the precipitate showed an enrichment of Pr and Nd (Table 3). Also tested as a control were wild type phages without any LanM peptides or proteins displayed.

Results. Elemental analysis found that the REE were present in the pH 7 precipitate. Inspection of Pourbaix diagrams show that Nd(OH)₃ would form at pH 7 or higher. Nd(OH)₃ is highly insoluble in water. The presence of Nd in the precipitate is consistent with the precipitation of the Nd as the hydroxide. This result also suggests that the EF peptides and LanM protein tested on the phage cannot bind to the hydroxides of the REE. The elution of the REE at neutral pH from Lanmodulin protein and EF peptides was unexpected, but notable as acid was not needed.

The M13 phage without REE-binding peptides or proteins displayed had some innate affinity for Pr and Nd. A similar result was found for Bacillus spores (ref. 5). However, the phage expressing the REE-binding peptides or LanM on them still had higher mass percentages of REE relative to the controls and to the starting material.

It is not believed that dialysis bags made of cellulose have been used previously to purify REE with biomaterials. The dialysis bag retains the phage. The dialysis bag is porous, but has a molecular weight (MW) cutoff of 3500 Da, preventing bacteria and other unwanted material from entering into the bag and damaging the biomaterials while allowing REE to pass through and bind to the phage.

The results show that the EF-P8 display phage which had the EF3 (SEQ ID NO: 6] or EF3 K94W peptide (SEQ ID NO: 7) displayed 2,700 times had a higher mass percentage of Nd and also Pr relative to the starting material and to the controls. This is consistent with high density metal binding.

TABLE 3
Elemental analysis of solutions and precipitates recovered with phage materials.

| | | | | | Nd(recovered)/ |
|---|---|---|---|---|---|
| State | Sample | Fe | Pr | Nd | Nd(control) |
| Starting Material | NdFeB Powder F1C | 66.6239 | 9.7973 | 22.3452 | |
| Solution | Starting Material | 11.5434 | 1.0184 | 4.4882 | |
| Solution | Starting Material | 33.9 | 2.61 | 5.6 | |
| pH 7 precipitate | Phage Tail Control | 28.3 | 3.5 | 7.17 | 1.00 |
| pH 7 precipitate | M4 (Deletion Mutant) | 32.7 | 2.88 | 7.86 | 1.10 |
| pH 7 precipitate | M5 | 34.527 | 3.2599 | 9.4317 | 2.10 |
| pH 7 precipitate | M5 | 36.3398 | 2.7187 | 9.8594 | 1.38 |
| pH 7 precipitate | Capsid Control | 39.7843 | 3.8774 | 10.573 | 1.00 |
| pH 7 precipitate | P8 EF3 Peptide | 40.6823 | 3.3465 | 8.5246 | 0.81 |
| pH 7 precipitate | P8 EF3 K94W | 46.6131 | 5.2574 | 12.1022 | 1.14 |
| | Peptide | | | | |

The elution of the REE as metal hydroxides at neutral pH was unexpected and not obvious. Many have thought that REE's could only be eluted at low pH (see ref. 2, eluting at <pH 2.5) which is typically damaging to biomaterials and could prevent their re-use. Lanmodulin binds REE very tightly making the removal of the REE from the protein difficult. Lanmodulin has picomolar affinity for REE even at low pH (see ref. 3). Dong, et al used dilute HCl pH<2.5 to elute REE from Lanmodulin that had been immobilized to microbeads (see ref. 2). The use of large volumes of strong acid is potentially hazardous. Thus, the elution of REE hydroxides at neutral pH yields a facile purification with minimal hazards. The precipitated REE hydroxides can be collected by centrifugation or filtering.

### Additional REE-Binding Peptides

Using standard techniques, additional recombinant phages were prepared in order to develop candidate peptide sequences for binding to REE.

A number of colonies were selected from plates to prepare monoclonal phage for a fluorescent protein competition assay using a cyan-yellow fluorescent protein FRET substrate, termed CFP-EF3-YFP and having the following sequence of amino acid residues:

(SEQ ID: 13)
MGSSHHHHHHSSGLVPRGSHMVSKGEELFTGVVPILVELDGDVNGHKF
SVSGEGEGDATYGKLTLKFICTTGKLPVPWPTLVTTLTWGVQCFARYP
DHMKQHDFFKSAMPEGYVQERTIFFKDDGNYKTRAEVKFEGDTLVNRI
ELKGIDFKEDGNILGHKLEYNAISDNVYITADKQKNGIKANFKIRHNI
EDGSVQLADHYQQNTPIGDGPVLLPDNHYLSTQSALSKDPNEKRDHMV
LLEFVTAAGITLSMDELYKGDPDNDGTLDKKEMVSKGEELFTGVVPIL
VELDGDVNGHKFSVSGEGEGDATYGKLTLKLICTTGKLPVPWPTLVTT
LGYGLQCFARYPDHMKQHDFFKSAMPEGYVQERTIFFKDDGNYKTRAE
VKFEGDTLVNRIELKGIDFKEDGNILGHKLEYNYNSHNVYITADKQKN
GIKANFKIRHNIEDGGVQLADHYQQNTPIGDGPVLLPDNHYLSYQSAL
SKDPNEKRDHMVLLEFVTAAGITLGMDELYKLEDPAANKAITLGMDEL
YK

Phage supernatants that showed the shift of IC50 curves in an in vitro competition assay relative to controls were selected and listed Table 4 below showing traits of either binding (“B”) or not binding (“N”) to Neodymium (Nd)/Dy (Dysprosium).

TABLE 4
Displayed peptide sequences for REE binding.

| | Nd | Dy |
|---|---|---|
| Displayed peptide sequences | Binding | Binding |
| TWFLGSCLSMTG (SEQ ID NO: 14) | B | N |
| GGVVPVSKYMDE (SEQ ID NO: 15) | B | N |
| VGVCCAWFRNFG (SEQ ID NO: 16) | B | B |
| HDTTVCSGSLAL (SEQ ID NO: 17) | N | B |
| LTWDIDDCPAPG (SEQ ID NO: 18) | B | B |
| LSKFLMGFFWIL (SEQ ID NO: 19) | B | B |
| LILVSELHSSRL (SEQ ID NO: 20) | N | B |
| SSSGSNLVVFEY (SEQ ID NO: 21) | B | N |
| FFAASLFSKNRY (SEQ ID NO: 22) | B | N |
| FIMLAHWLLWIL (SEQ ID NO: 23) | B | B |
| FLFSMYRVFGPQ (SEQ ID NO: 24) | B | N |
| LFRYFLGFTFYF (SEQ ID NO: 25) | N | B |
| WKVSSFFVDGFC (SEQ ID NO: 26) | B | N |
| MSKWFDSFRSEG (SEQ ID NO: 27) | B | N |
| MLSFLREFPDAF (SEQ ID NO: 28) | N | B |
| YSSLKFLVVSGA (SEQ ID NO: 29) | B | B |
| LMAWYCWYMGVR (SEQ ID NO: 30) | N | B |
| FVVGQSONTQFA (SEQ ID NO: 31) | N | B |
| HIMGVFSLSVGS (SEQ ID NO: 32) | N | B |
| STFRLIARVLRW (SEQ ID NO: 33) | N | B |
| HSILGISGVSNV (SEQ ID NO: 34) | N | B |
| ENLVNYCWCMSI (SEQ ID NO: 35) | B | B |
| RKLGGMIGYWTS (SEQ ID NO: 36) | B | B |
| ASNELKGSSMFG (SEQ ID NO: 37) | B | B |
| FCIPGCLNFFIV (SEQ ID NO: 38) | N | B |
| LCLFLRLVDVSL (SEQ ID NO: 39) | B | B |
| YSSLKFLVVSGA (SEQ ID NO: 40) | B | B |
| KMVPWGSVIWFG (SEQ ID NO: 41) | B | B |
| VHVFMRRFGVLW (SEQ ID NO: 42) | N | B |
| YTSGVRYVLWWW (SEQ ID NO: 43) | N | B |
| CYLCRAFPNFGS (SEQ ID NO: 44) | N | B |
| GSRLLACDFFLY (SEQ ID NO: 45) | N | B |
| ILLMSCRGCVKS (SEQ ID NO: 46) | B | B |
| TAPHCSLGARNI (SEQ ID NO: 47) | N | B |
| TLEFLLSPCQLA (SEQ ID NO: 48) | N | B |
| YRSSLHLFSSLS (SEQ ID NO: 49) | N | B |
| WTFDSLDSSTCG (SEQ ID NO: 50) | B | B |
| LQFILCAKGDDV (SEQ ID NO: 51) | B | B |

B represents binding, N represents no binding.

Additional peptide sequences were tested for selective binding to Nd as opposed to binding to Pr (Praseodymium) by washing the Nd-charged column with Pr prior to elution. These are shown below in Table 5.

TABLE 5
Peptides with Nd-specific binding.

| | | Nd | Pr |
|---|---|---|---|
| Name | sequence | binding | binding |
| B10 | QCMRLSWVNNCV (SEQ ID NO: 52) | B | N |
| D10 | HFFLLANTTVVR (SEQ ID NO: 53) | B | N |
| E7 | FYGLWFYRMSGS (SEQ ID NO: 54) | B | N |
| G9 | LDIIKRFCLGMH (SEQ ID NO: 55) | B | N |
| G11 | MGSVRSDRYQFV (SEQ ID NO: 56) | B | N |
| H10 | NITLVKSILVDR (SEQ ID NO: 57) | B | N |
| H11 | FHTSSEFLSSVC (SEQ ID NO: 58) | B | N |
| H12 | TGNIHGSGLLLR (SEQ ID NO: 59) | B | N |

A fluorescent competition assay was also used to select peptides displayed on P8 in the phage that bind to Yb. These peptides are seen below in Table 6.

TABLE 6
Peptides with Yb-binding potential.

| Name | sequence |
|---|---|
| YbB4 | DTNNDGEYEGDEL (SEQ ID NO: 60) |
| YbC4 | DTNNDGLYEGDEL (SEQ ID NO: 61) |
| YbD4 | DTNNDGGYEGDEL (SEQ ID NO: 62) |
| YbE4 | DTNNDGRYEGDEL (SEQ ID NO: 63) |
| YbA9 | DTNNDGSYEGDEL (SEQ ID NO: 64) |
| YbA11 | DTNNDGTYEGDEL (SEQ ID NO: 65) |
| YbD11 | DTNNDGAYEGDEL (SEQ ID NO: 66) |
| YbB12 | DTNNDGVYEGDEL (SEQ ID NO: 67) |
| YbH12 | DTNNDGIYEGDEL (SEQ ID NO: 68) |

Phage display was used to search for REE-binding phage with a competition assay. A tryptophan residue was found to be preferred in Tb-specific peptides in a survey of the PDB for REE-binding peptide sequences. However, as the Tb-peptides became more specific for Tb, they also became more specific for Yb. Thus, the W was randomized in another phage library and then screened. Tb-binding peptides have been reported in the literature with Kd ranging between 2 nM to 6 uM (see ref. 9). Two of thee phage P8-peptide sequences are provided below in Table 7.

| Name | Sequence |
|---|---|
| TB1 | YIDTNNDGWYEGDELLA (SEQ ID NO: 69) |
| TB2 | ACVDWNNDGWYEGDECA (SEQ ID NO:70) |

These REE-peptide displaying phage (displayed on P8) were tested for Tb-binding. Using 100 mg of each phage in a dialysis bag, the amount of metal eluted after 12 cycles of bind-wash-elute-wash ranged from 10-11.7 mg using 2 mg/mL solutions of Tb in 50 mM MES pH 6.0. While yields were comparable, differences in purity were apparent. Notably, as seen in Table 7 below, the TB2 peptide produced completely pure product to the limit of detection. The purity was higher than that of the starting material. The binding was down in approximately 40 mL volumes of aqueous solutions.

TABLE 7
Efficacy of Tb-peptide phage in bio-purifying Tb using dialysis bags.

| | Amount | Tb-Oxalate | | | | | | |
|---|---|---|---|---|---|---|---|---|
| | Eluted at | from | Na+ | K+ | Cl− | Ti | Ta | Tb³⁺ |
| | pH 1.4 (mg) | Eluate (mg) | (%) | (%) | (%) | (%) | (%) | (%) |
| Starting | | | 0.0218 | | | 0.3243 | | 99.6552 |
| Material | | | | | | | | |
| P8 | 10.4 | 10.7 | | 0.1766 | 0.4018 | | | 99.4215 |
| control | | | | | | | | |
| EF3 | 11.7 | 12.8 | | 0.1624 | 0.5315 | 1.2379 | | 98.0682 |
| TB1 | 11.7 | 8.2 | | | | 1.1067 | 2.0796 | 96.8137 |
| TB2 | 10.9 | 12.5 | | | | | | 100.0000 |

### Further Embodiments

The acidic conditions used for REE binding could potentially vary from pH 2.5 to pH 6. The acidic conditions for REE elution from phage could vary from pH 1 to 2.5. For example, elution can occur with 50 mM glycine pH 1.4 buffer, which was effective for Nd and Tb. These were then precipitated as oxalates. Glycine is an amino acid and is considered a “green” and environmentally friendly biochemical. Oxalates and other carbonates can be made after elution and removal of the dialysis bag from the elution buffer (50 mM Glycine pH 1.4) at room temperature (20° C.). The neutral conditions used for REE elution could potentially vary from pH 6 to >pH 10. In some cases it may be desirable to limit the elution pH to a pH of about 8 or less in order to prevent the formation of hydroxides.

It is also expected to be able to generate make chlorides (halide) and sulfate salts of the REE.

A variety of suitable dialysis membranes could be used, including those of cellulose or other materials. While the example used a MW cutoff of 3500 Da, membranes passable to other sizes of molecules would work so long as they block passage of the engineered phage biomaterial while allowing passage of the REE.

The biomaterials invented here can be replicated using helper phage or by itself for the recombinant M13 phage with exogenous gene insert. This sustainable resource can be produced from bacterial media. The requirement for helper phage may limit or reduce the risk of an accidental spill into the environment. Recombinant M13 phages can also be biomanufactured at an industrial scale.

It is expected that the peptide alones can also be used as a lanthanide binding ‘tag’ and/or included in non-phage proteins. The peptides themselves could be used to selectively attach metals to other proteins similar to the use of Lanthanum binding Tags (LBT) in X-ray crystallography or for imaging.

### Advantages

This technique provides a relatively benign approach to purifying REE without requiring harsh or toxic solvents, but instead is done in aqueous solution. It is amenable to purification from various feedstocks and leachates. The phage used can be easily produced via standard biological culture techniques. Moreover, there is no need for costly inducers of protein expression such as isopropyl ß-d-1-thiogalactopyranoside (IPTG), nor costly antibiotics. Glycine buffers (an amino acid) were compatible. The high metal to carbon ratio for the small P8 protein also minimized the amount of material needed for larger scale REE purification. Other biomaterials such as bacteria and fungi have phospholipid membranes and other extraneous proteins. They also require continual growth and feeding with a carbon and nitrogen source. The REE-binding phage which are comprised substantially of P8 could densely bind REE without the need of maintaining an aerated culture or adding glucose as would be needed with a living organism such as a bacterium or fungus.

Furthermore, tested phage were stable at pH 3.6 for long periods at room temp and also showed good tolerance to the pH 1.4 buffer with 20 minutes of exposure.

The process can be accomplished using minimal power (e.g., electricity), and without stirring, pumping, or the like, hence the term “passive recovery.”

The use of a dialysis membrane avoids the need for binding a biomaterial to a solid support such as a resin or bead and allows for easy removal of the bound material. However, due to the use of a phage particle, the technique is operable without any need for immobilization to a solid matrix. The leachates and REE-containing eluates are not sullied by the biomaterial, and 100% pure REE can be achieved.

Acid can also hydrolyze, denature, and aggregate proteins thus other biomaterials may or may not withstand pH<2.5 preventing their reuse in subsequent rounds of purification. Reuse of biomaterials reduces their cost. In the above exemplary protocol, the REE bound at pH 3.7 and eluted at pH 7. This suggests that the phage may be able to be re-used in multiple cycles of purification. In the aforementioned examples, 12 cycles were carried out. Based on the Pourbaix diagram (see ref. 7), Nd is predicted to stay in solution between pH 1 to pH 6. Iron oxides readily precipitate in this pH range. Thus, Nd₂Fe₁₄ B solutions can be partially cleared of Fe oxides by raising the pH to 3.7 or higher. These M13 phage were stable at pH 1 to 9 (see Folia Microbiol (Praha). 2011; 56(3): 191-200) and also allowed low pH elution in 50 mM Glycine pH 1.4 buffer. Oxalic acid could be added to the elution tube to precipitate the water insoluble REE-oxalate and further wash the material to remove other soluble ions.

### Concluding Remarks

All documents mentioned herein are hereby incorporated by reference for the purpose of disclosing and describing the particular materials and methodologies for which the document was cited.

Although the present invention has been described in connection with preferred embodiments thereof, it will be appreciated by those skilled in the art that additions, deletions, modifications, and substitutions not specifically described may be made without departing from the spirit and scope of the invention. Terminology used herein should not be construed as being “means-plus-function” language unless the term “means” is expressly used in association therewith.

### REFERENCES

1. T. Sawada, Filamentous virus-based soft materials based on controlled assembly through liquid crystalline formation. Polymer Journal 49, 639-647 (2017).
2. Z. Dong et al., Bridging Hydrometallurgy and Biochemistry: A Protein-Based Process for Recovery and Separation of Rare Earth Elements. ACS Central Science 7, 1798-1808 (2021).
3. J. A. Cotruvo, Jr., E. R. Featherston, J. A. Mattocks, J. V. Ho, T. N. Laremore, Lanmodulin: A Highly Selective Lanthanide-Binding Protein from a Lanthanide-Utilizing Bacterium. Journal of the American Chemical Society 140, 15056-15061 (2018).
4. T. Sawada, M. Asada, T. Serizawa, Selective Rare Earth Recovery Employing Filamentous Viruses with Chemically Conjugated Peptides. Chemistry Select 1, 2712-2716 (2016).
5. W. L. Dong, S.; Camilleri, E.; Korza, G.; Yankova, M.; King, S. M.; Setlow, P., Accumulation and Release of Rare Earth Ions by Spores of Bacillus Species and the Location of These Ions in Spores. Appl Environ Microbiol 85, (2019).
6. E. C. Cook, E. R. Featherston, S. A. Showalter, J. A. Cotruvo, Jr., Structural Basis for Rare Earth Element Recognition by Methylobacterium extorquens Lanmodulin. Biochemistry 58, 120-125 (2019).
7. M. O. Tanaka, T; Kazuya, K.; Hirokazu, N.; Oishi, T., Handbook on the Physics and Chemistry of Rare Earths. J. C. G. P. Bunzli, V. K., Ed., (2013), vol. 43.
8. US Patent Publication No 2021/0189521 “Metal-binding protein and use thereof”
9. Nitz et al. “A powerful combinatorial screen to identify high-affinity terbium(III)-binding peptides” Chembiochem. 2003 Apr. 4; 4(4):272-6. doi: 10.1002/cbic.200390047.

What is claimed is:

1. A method of capturing a rare earth element, the method comprising:
providing a construct comprising an M13 phage displaying one or more metal-binding peptide sequences on capsid proteins thereof;
contacting, under acidic conditions, the construct with a solution comprising a rare earth metal (REE), wherein the construct and the solution are initially separated by a dialysis membrane;
allowing the REE to pass through the membrane and bind to the construct; and
eluting the REE from the construct.

2. The method of claim 1, wherein the construct is not bound to a solid surface.

3. The method of claim 1, wherein said peptide sequences are selected from the group consisting of SEQ ID NOs: 9 to 12, inclusive.

4. The method of claim 3, wherein at least one of the peptide sequences is SEQ ID NO: 12.

5. The method of claim 1, wherein the M13 phage comprises SEQ ID NO: 6 or SEQ ID NO: 7.

6. The method of claim 1, wherein the M13 phage comprises a sequence selected from the group consisting of SEQ ID NOs: 9 to 70, inclusive.

7. The method of claim 1, wherein the M13 phage comprises SEQ ID NO: 70.

8. The method of claim 7, wherein said REE is Tb.

9. A biological construct for capturing rare earth elements, the construct comprising:
an M13 phage displaying one or more metal-binding protein sequences on capsid proteins thereof.

10. The construct of claim 9, wherein said sequences are selected from the group consisting of SEQ ID NOs: 9 to 12, inclusive.

11. The construct of claim 10, wherein at least one of the sequences is SEQ ID NO: 12.

12. The construct of claim 9, comprising an M13 phage expressing SEQ ID NO: 6 or SEQ ID NO: 7

13. The construct of claim 9, wherein said sequences are selected from the group consisting of SEQ ID NOs: 9 to 70, inclusive.

14. The construct of claim 9, wherein said one or more metal-binding protein sequences comprises SEQ ID NO: 70.

15. A biological construct comprising one or more amino acid sequences selected from the group consisting of SEQ ID NOs: 9 to 70, inclusive.

16. The biological construct of claim 16, comprising SEQ ID NO: 13.
